(19) United States
(12) Patent Application Publication
(10) Pub. No.: US 2015/0289813 A1
(43) Pub. Date: Oct. 15, 2015

(54) System and Method for the Biological Diagnosis of Post-Traumatic Stress Disorder: PTSD Electronic Device Application

(71) Applicant: Eugene Lipov, Hoffman Estates, IL (US)

(72) Inventor: Eugene Lipov, Hoffman Estates, IL (US)

(21) Appl. No.: 14/687,029

(22) Filed: Apr. 15, 2015

Related U.S. Application Data
(60) Provisional application No. 62/000,060, filed on May 19, 2014.
(60) Provisional application No. 61/979,599, filed on Apr. 15, 2014.

(51) Int. Cl. A61B 5/00; A61B 3/11; A61B 5/16; A61B 5/0484; A61B 5/04; A61B 5/0488; A61B 5/1455; A61B 3/14; A61B 5/0205

(52) U.S. Cl. CPC A61B 5/4884; A61B 3/145; A61B 3/112; A61B 5/165; A61B 5/0205; A61B 5/04012; A61B 5/0488; A61B 5/6898; A61B 5/14552; A61B 5/6803; A61B 5/04842; A61B 5/04845; A61B 5/7246; A61B 2576/02; A61B 5/02438

(57) ABSTRACT

A system and method for the biological diagnosis of Post-Traumatic Stress Disorder (PTSD) comprises an electronic device equipped with a built-in or attachable camera, built-in or attachable flash or controllable light source, and a software application. The software application includes a method that records and monitors the diameter of an individual's pupil prior to and after the application of light, using the camera and flash in communication with the electronic device. In another embodiment, the method includes the use of emotionally-charged visual stimuli to increase the accuracy of the diagnosis. In still another embodiment, a heart rate monitor in communication with the electronic device is used to monitor the individual's heart rate variability to further increase the accuracy of the diagnosis. In still another embodiment, the method also measures the auditory startle response. The method analyzes the data collected and determines the likelihood the individual suffers from PTSD.

This application claims priority to provisional application Ser. No. 62/000,060 filed May 19, 2014, to the extent allowed by law and to provisional application Ser. No. 61/979,599 filed Apr. 15, 2014, to the extent allowed by law.

### BACKGROUND OF THE INVENTION

1. Field of the Invention

The present invention relates to a system and method for the biological diagnosis of Post-Traumatic Stress Disorder, and more particularly, to a Post-Traumatic Stress Disorder biological diagnostic method implemented by a system that utilizes an electronic device, such as a computer or a mobile device, and attached peripherals, such as a camera, flash, heart rate monitor, digital monitor, ear lobe monitor, electroencephalogram (EEG) monitor, and bispectral index (BIS) monitor (another form of EEG), as well as additional add-ons. Further, the same device may be used to monitor the progress of Post-Traumatic Stress Disorder treatment.

2. Description of the Prior Art

Post-Traumatic Stress Disorder (PTSD) is a devastating and complex pathological anxiety condition that is characterized by severe distress and impairment in mental and physical functioning. Symptoms typically include intense anxiety, hyper-arousal, flashbacks, and sleep disturbances. The impact and consequences for individuals diagnosed with PTSD include depression, substance abuse, violence, inability to maintain intimate relationships, inability to maintain parental relationships, suicide, and premature mortality. PTSD is also a public health dilemma because nearly 80% of residents experience traumatic events in their lifetime. Women are twice as likely to develop PTSD symptoms as men due to the prevalence of sexual assaults. Further, the ever increasing rate of military suicides has been linked in many instances to PTSD.

Individuals impacted with PTSD require swift diagnosis and medical intervention. Usually, PTSD is diagnosed by psychiatrically trained professionals using questionnaires. The use of biological methods, such as functional magnetic resonance imaging (FMRI) and other scanners, have been shown to diagnose PTSD with up to 90% accuracy. Other biological evaluations of PTSD are functional magnetic resonance imaging (FMRI) and magnetoencephalography (MEG). FMRI is a functional neuroimaging procedure using MRI technology that measures brain activity by detecting associated changes in blood flow. The technique relies on the fact that cerebral blood flow and neuronal activation are coupled. When an area of the brain is in use, blood flow to that region also increases. MEG is a functional neuroimaging technique for mapping brain activity by recording magnetic fields produced by electrical currents occurring naturally in the brain, using very sensitive magnetometers. However, due to the extremely high cost of FMRI and MEG scanners, the alternative biological methods of diagnosis of PTSD are urgently needed, especially biological diagnostic methods of PTSD for use in electronic devices.

Current PTSD diagnostic methods include heart rate variability, Clinician-Administered PTSD Scale for DSM-5 (CAPS-5), and neuroimaging. Heart rate variability has been used for the evaluation of PTSD, however, it has limited effectiveness and is still controversial. The heart rate variability approach utilizes the fact that under stress the sympathetic system is over activated, which takes away variability of the heart beat-to-beat variation. This is an explanation via the polyvagal theory.

CAPS-5, which is currently considered a “gold standard,” is a 30-item structured interview that corresponds to the Diagnostic and Statistical Manual of Mental Disorders, 5th Edition, (DSM-5) criteria for PTSD. CAPS-5 was designed to be administered by clinicians and clinical researchers who have a working knowledge of PTSD, but can also be administered by properly trained paraprofessionals. The full CAPS-5 interview takes 45-60 minutes to administer, is under 60% accurate, and is highly subjective. Another way to diagnose PTSD with questionnaires has been PCL that has a reported accuracy of under 60%. PCL is a standardized self-reporting rating scale for PTSD comprising seventeen items that correspond to the key symptoms of PTSD.

Neuroimaging studies for the diagnosis of PTSD are under development. Reviews were conducted of all functional and structural neuroimaging studies of subjects with PTSD. Studies were identified using general medical and specific traumatic stress databases, paper searches of current content, and other secondary sources. The most replicated structural finding is hippocampal volume reduction, which may limit the proper evaluation and categorization of experience. Replicated localized functional changes include increased activation of the amygdala after symptom provocation, which may reflect its role in emotional memory, and decreased activity of Broca's Area at the same time, which may explain the difficulty patients have in labeling their experiences. Neuroimaging has accuracy close to 80-90%, but is very expensive to operate and has limited availability. As such, a need for a swift, accurate, and inexpensive method for the biological diagnosis of PTSD is desperately needed at this time to contain the tide of misery due to PTSD.

A group of students in Poland have developed a mobile device application that monitors iris response for monitoring drug use. The application can determine whether an individual took drugs or other substances. The application sets off the flash on the mobile device, then measures the size of the individual's pupil and iris and checks the whiteness of the eye. Although a blood test is needed to confirm drug use 100%, the application proved to be very effective.

The present invention was developed as a result of the extensive work by the inventor with stellate ganglion block (SGB) known to reduce symptoms of PTSD via reduction of sympathetic tone. He noticed the patients that had done well also reported a clearer vision. His explanation of this was a more constricted pupil. The basic explanation is that a tiny hole is essentially a narrow aperture, which increases the depth of field of vision by the way in which light has to travel through the aperture, thus making more objects appear to be in focus to the eyes. He further realized that the pupil is dilated with increased sympathetic tone.

The present invention was also developed as a result of the extensive work by the inventor on evaluating the sympathetic system for the treatment of PTSD using SGB. Over ten years, the startle response, and extinction of the response, has been used to evaluate the diagnosis of PTSD. The approach requires a subject to receive multiple sound stimulations of 95 decibels and measuring the response of the heart rate increase as well as the eye blink reflex using the orbicularis oculi electromyography (EMG). Previous papers have speculated that changes in the brain due to PTSD can increase the susceptibility of the patient to have an increased startle reflex as demonstrated via EMG and heart rate change. FMRI, discussed above, assumes changes in the brain similar to the ones discussed above, such as over sensitivity or activation of the amygdala. The present invention uses the above discoveries by integrating them into a practical application. The base line measures after sound stimulation are likely to demonstrate PTSD in a convenient way. However, the “signal of PTSD,” or how easy it can be diagnosed, can be increased by stimulation of the brain, presumably the amygdala, by the use of emotional images, which are used to activate the amygdala for FMRI.

A primary object of the present invention is to provide a fast, inexpensive, and accurate method for the biological diagnosis of PTSD or the risk of PTSD presence, as well as providing a follow up response to treatments and possibly to diagnose a danger to self-harm or harm to others.

It is another object of the present invention to provide a device that implements the method for the biological diagnosis of PTSD.

### SUMMARY OF THE INVENTION

The system and method for the biological diagnosis of PTSD of the present invention comprises an electronic device, such as a computer or mobile device, equipped with a built-in camera or an attachable camera, a flash and/or other controllable light sources, and a software application for the analysis of data. The software application includes a method to measure a lessened or changed pupillary constrictive response to light, using either still images or video recording, that indicates that the individual suffers from PTSD. The accuracy of the diagnosis is increased by utilizing emotionally provocative visual stimuli, such as images or video of a mutilated body. To further increase the accuracy of using pupillary response, the individual's heart rate variability is monitored simultaneously, using a heart rate monitor, such as a digital finger monitor, ear lobe monitor, or the like.

The system of a first embodiment of the present invention comprises a device equipped with a built-in or attachable camera. The device utilizes the built-in or attachable camera to record the size of the individual's pupil and the pupillary response to light. In a second embodiment, the device includes a heart rate monitor to measure the individual's heart rate variability simultaneously while measuring the pupillary response. A secondary camera, diode, or any other heart rate monitor can be attached to an input port in the device. Finally, the hardware and software may interact with a virtual psychologist or other medical professional in the computer system to help with the diagnosis and follow up of the patient.

The system of a third embodiment of the present invention comprises an electronic device, such as a computer or mobile device, equipped with a built-in camera or an attachable camera, a head set, and a software application for the analysis of data. The headphones are used to play a loud sound, such as a sound at 95 decibels, for the individual in order to measure the individual's auditory startle response. The device then utilizes the built-in or attachable camera to record the size of the individual's pupil and the pupillary response to the loud sound and measures the individual's heart rate variability simultaneously while measuring the pupillary response. A secondary camera, diode, or any other heart rate monitor can be attached to an input port in the device. The individual is then shown emotionally provocative visual stimuli and the pupillary response and the heart rate variability is measured again.

### BRIEF DESCRIPTION OF THE DRAWINGS

The system and method of the present invention is further described with reference to the accompanying drawings in which:

FIG. 1 is a system overview of a first embodiment of the system and method of the present invention.

FIG. 2 is a screenshot of a mobile device of a second embodiment of the system and method of the present invention displaying a view of a built-in camera focusing on an individual's pupil.

FIG. 3 is a flow diagram of steps one through four of the method of the first and second embodiments of the present invention.

FIG. 4 is a flow diagram of steps five through eight of the method of the first and second embodiments of the present invention.

FIG. 5 is a flow diagram of steps nine through eleven of the method of the first and second embodiments of the present invention.

FIG. 6 is a flow diagram of a third embodiment of the system and method of the present invention.

FIG. 6A is a flow diagram of a fourth embodiment of the system and method of the present invention.

FIG. 7 is a perspective view of a first head set used in the third embodiment of the system and method of the present invention.

FIG. 8 is a perspective view of the first head set worn by an individual in the third embodiment of the system and method of the present invention.

FIG. 9 is a perspective view of a second head set using in the third embodiment of the system and method of the present invention.

FIG. 10 is a perspective view of the second head set worn by an individual in the third embodiment of the system and method of the present invention.

### DETAILED DESCRIPTION OF THE ILLUSTRATED EMBODIMENTS

The system and method for the biological diagnosis of PTSD of an embodiment of the present invention is based on the principle that the pupillary light reflex is related to the functions of the sympathetic nervous system. The autonomic nervous system manages involuntary functions such as pupil size, as well as heart rate and perspiration, and controls the movement of the iris in relation to light exposure. The autonomic nervous system is composed of three main sub-systems: the parasympathetic nervous system, the sympathetic nervous system, and the enteric nervous system. The sympathetic nervous system performs three main functions: regulating the cardiovascular system, regulating body temperature, and implementing the “Fight or Flight” reaction. Stimulation of the sympathetic sub-system, that controls “Fight or Flight” reactions when the body is under stress, causes the pupils to dilate. Pupillary light reflex controls the diameter of the pupil in response to the intensity of light that falls on the retina of the eye and in bright light causes the pupils to constrict. Individuals suffering from PTSD are less responsive to light, causing their pupils to remain open longer than the pupils of an individual without PTSD.

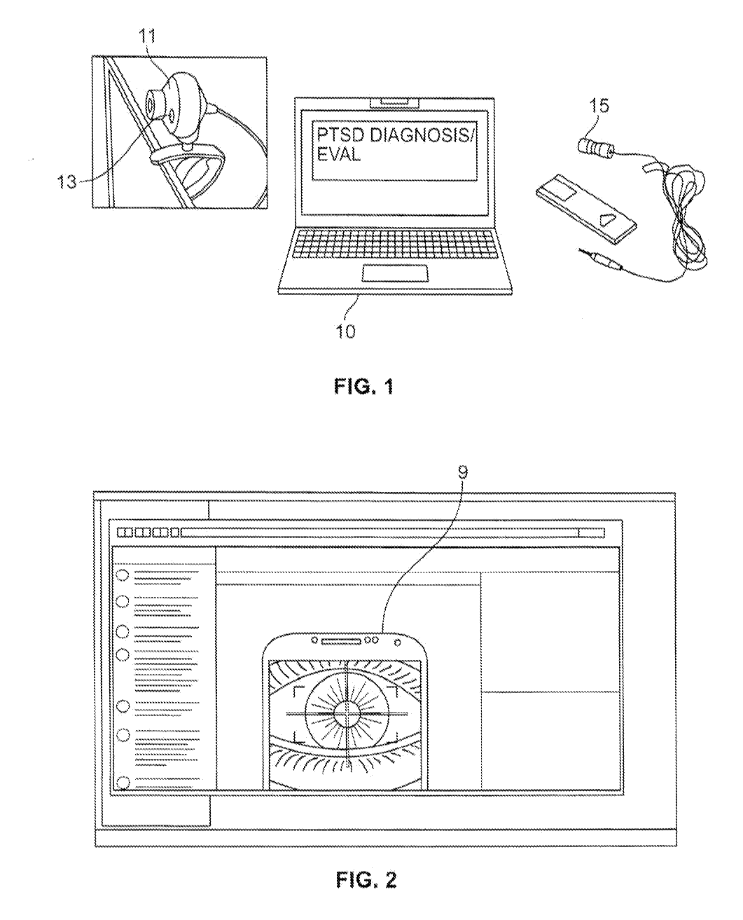

A first embodiment of the system and method of the present invention, shown in FIG. 1, comprises an electronic device 10 equipped with a built-in camera 11 or an attachable camera 11, a flash and/or other controllable light sources 13, and a software application (not shown) for the analysis of data. The system and method of the present invention can also include a heart rate monitor 15, such as the ear lobe monitor 15 shown in FIG. 1. The software application includes a method that comprises the measurement of a lessened pupillary constrictive response to light that indicates that the individual suffers from PTSD. The camera 11 of an electronic device 10 is used to record the pupil of an individual. In a second embodiment, the electronic device 10 can comprise a mobile electronic device 9, as shown in FIG. 2.

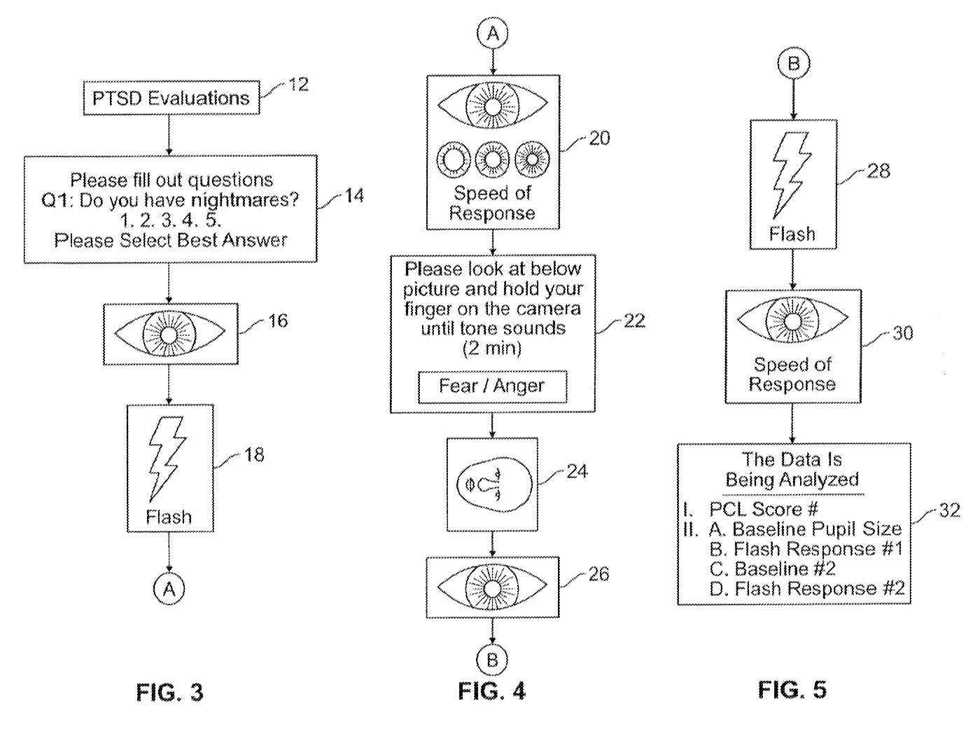

Referring to FIG. 3, the method begins the PTSD evaluation 12 by having the individual answer a questionnaire 14. After the individual has completed the questionnaire, the camera 11 in or attached to the electronic device 10 measures the diameter of the pupil prior to the application of light 16. The individual is then flashed 18 using the flash 13 in or attached to the electronic device 10 and immediately thereafter the camera 11 measures the speed of the pupil's response 20, shown in FIG. 4. The method calculates the difference between the size of the diameter of the pupil prior to the application of light and after the application of light. The electronic device 10 may also record the response of the pupil over time, such as the motion of the pupil, not just the static size of the pupil.

The accuracy of the diagnosis is increased by showing the individual emotionally-charged visual stimuli 22, such as images or video of a mutilated body 24, when performing the method. To further increase the accuracy, the individual's heart rate variability (HRV) is monitored simultaneously 22 using a built-in or an attachable heart rate monitor 15, such as a pulse oximeter probe, a digital finger monitor, an ear lobe monitor, and the like. The camera 11 in the electronic device 10 again measures the diameter of the pupil prior to the application of light 26.

The individual is then flashed 28, shown in FIG. 5, again using the flash 13 attached to the electronic device 10 and immediately thereafter the camera 11 measures the speed of the pupil's response 30. The method calculates the difference between the size of the diameter of the pupil prior to the application of light and after the application of light. As stated above, the electronic device 10 may also record the response of the pupil over time, such as the motion of the pupil, not just the static size of the pupil.

HRV is the physiological phenomenon of variation in the time interval between heart beats. HRV is measured by the variation in the beat-to-beat interval. Individuals suffering with PTSD have decreased HRV under conditions of acute time pressure, emotional strain, and elevated state anxiety. HRV has been shown to be reduced in individuals reporting a greater frequency and duration of daily worry.

The method analyzes the data 32, shown in FIG. 5, which can include the PCL score, the baseline pupil size at 16, the speed of response measured at 20, the baseline pupil size at 26, and the speed of response measured at 30. The method analyzes the change in pupillary diameter and, if available, the pupillary response to emotionally stressful visual stimuli and the individual's heart rate variability to determine the likelihood that the individual is suffering from PTSD.

Referring again to FIG. 1, the system of a first embodiment of the present invention comprises an electronic device 10, such as a computer, and attached peripherals, such as a camera 11, flash 13, heart rate monitor 15, such as a digital finger monitor and an ear lobe monitor, as well as additional add-ons. The electronic device 10 is equipped with a built-in or an attachable camera 11 that includes a software application that performs the method of the present invention. The electronic device 10 utilizes the camera 11 to record the size of the individual's pupil and the pupillary response to light shone in the individual's eye using the electronic device's 10 built-in or attachable flash 13. In a second embodiment, the electronic device 10 can comprise a mobile electronic device 9, as shown in FIG. 2. The software application analyzes the data, the diameter of the pupil prior to and after the application of light, and determines the difference in diameter and the likelihood that the individual is suffering from PTSD. In a second embodiment, the electronic device 10 includes a heart rate monitor 15 to measure the individual's heart rate variability simultaneously while measuring the pupillary response, thereby increasing accuracy because individuals suffering from PTSD have unsteady heart rates. A secondary camera, diode, or any other heart rate monitor 15 can be attached to an input port in the electronic device 10. The software application analyzes the data collected from the heart rate monitor 15 and includes this data in the evaluation of the likelihood that the individual is suffering from PTSD.

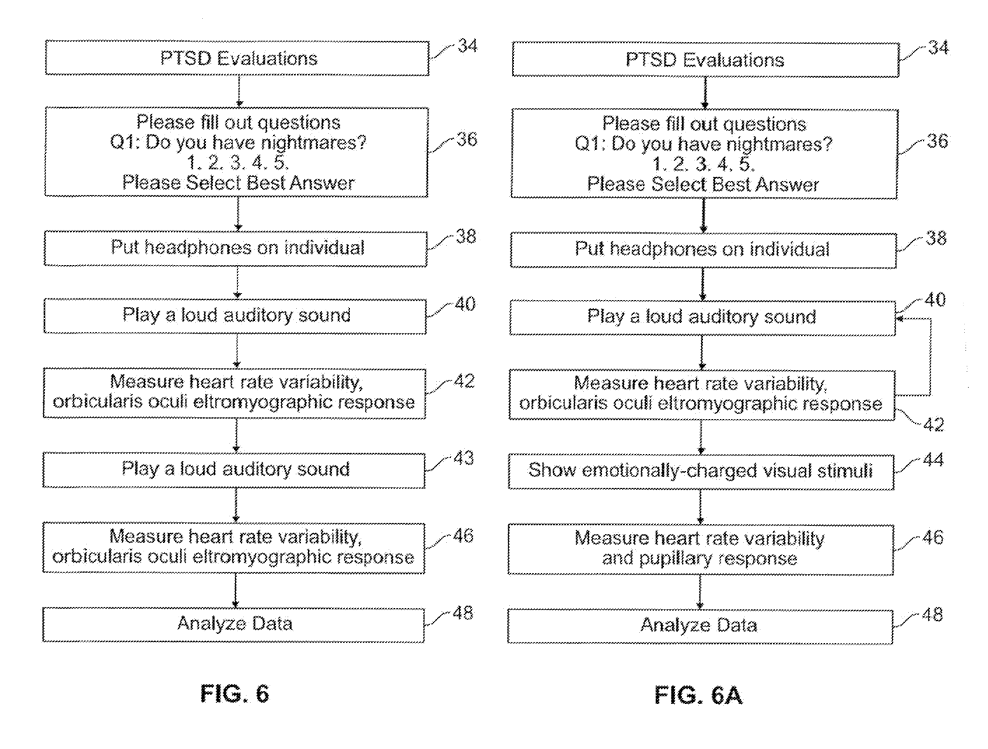

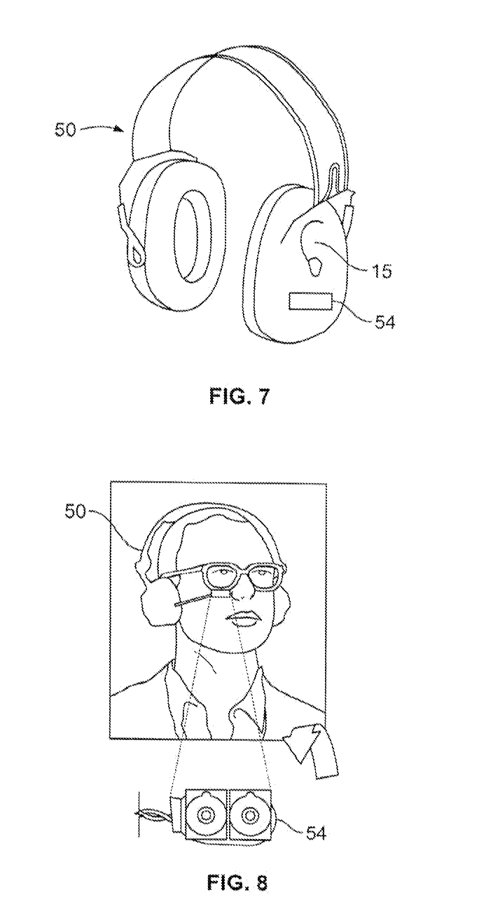

Referring to FIG. 6, in a third embodiment of the present invention the system measures the auditory startle response of the individual. The system comprises an electronic device 10 equipped with a built-in camera 11 or an attachable camera 11, a first head set 50 (FIG. 7, 8), and a software application (not shown) for the analysis of data. The head set 50 includes a heart rate monitor 15 (FIG. 7), such as a pulse oximeter probe for the heart rate evaluation, and electromyography (EMG) leads 54 (FIGS. 7, 8). The method of the third embodiment begins the PTSD evaluation 34 by having the individual answer a questionnaire 36. After the individual has completed the questionnaire, the individual is instructed to put headphones on 38 and a sudden, loud tone or noise, such as a sound of 95 decibels, is played for the individual 40. The EMG leads 54 measure the individual's orbicularis oculi (eye blink) electromyographic responses while simultaneously monitoring the individual's HRV 42 using the attached heart rate monitor 15. A second sudden, loud tone or noise, such as a sound of 95 decibels, is again played for the individual 43. The EMG leads 54 measure the individual's orbicularis oculi (eye blink) electromyographic responses again while simultaneously monitoring the individual's HRV 46 using the attached heart rate monitor 15. The method then analyzes the data 48, which can include the PCL score and the speed of response. The method analyzes the orbicularis oculi (eye blink) electromyographic responses to the auditory startle response and the individual's HRV related to the auditory startle response to determine the likelihood that the individual is suffering from PTSD.

Referring to FIG. 6A, a fourth embodiment of the present invention includes the steps of the third embodiment and additionally the method continues and the individual is then shown an emotionally-charged visual stimuli 44, such as images or video of a mutilated body. The camera 11 in or attached to the electronic device 10 measures the pupillary response of the individual while simultaneously monitoring the individual's HRV 46. The method then analyzes the data 48, which can include the PCL score, the baseline pupil size, and the speed of response. The method analyzes the orbicularis oculi (eye blink) electromyographic responses to the auditory startle response, the pupillary response to emotionally stressful visual stimuli, and the individual's HRV related to the auditory startle response and to the emotionally stressful visual stimuli to determine the likelihood that the individual is suffering from PTSD.

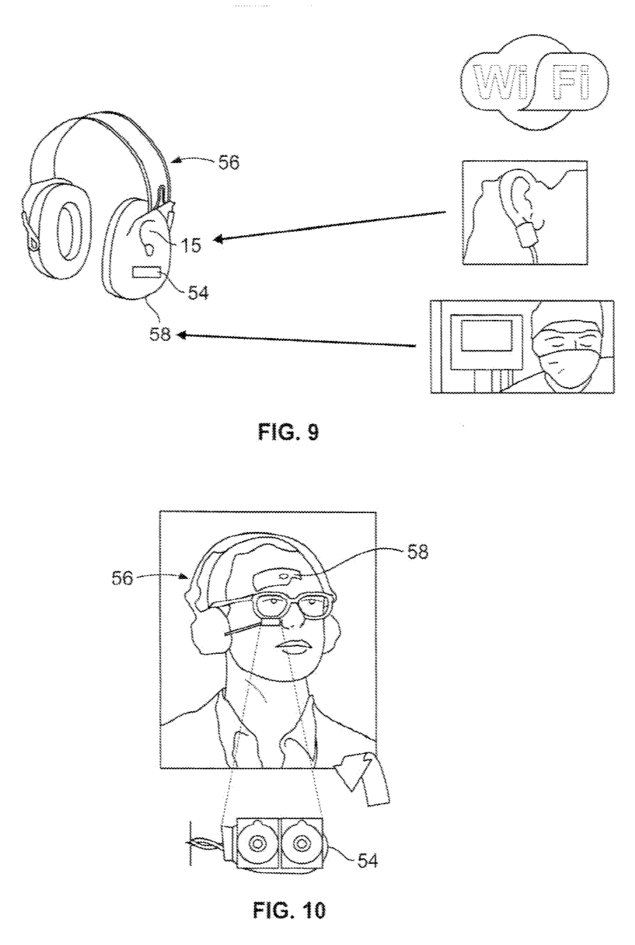

Alternatively, the system and method in the third embodiment and in the fourth embodiment can comprise a second head set 56 (FIG. 9, 10) that includes a heart rate monitor 15, such as a pulse oximeter probe for the heart rate evaluation, electromyography (EMG) leads 54, and bispectral index (BIS) leads 58, such as electroencephalogram (EEG) leads. The EMG leads 54 measure the individual's orbicularis oculi (eye blink) electromyographic responses while simultaneously monitoring the individual's HRV 42 using the attached heart rate monitor 15 and measuring the individual's depth of consciousness and monitoring the depth of anesthesia using the BIS leads 58, such as the EEG leads. The BIS leads 58 will give an electroencephalogram score between zero and 100. The bispectral index is a weighted sum of several electroencephalographic subparameters, including a time domain, frequency domain, and high order spectral subparameters. The BIS leads 58 provide a single dimensionless number. Electroencephalogram (EEG) changes have been shown to demonstrate the over activation of the brain in individual's suffering from PTSD. Further, SGB has been shown to deactivate the BIS monitor recording in normal volunteers.

In a fifth embodiment of the present invention, the head set 50, 56 can further include a conductance sensor 52 (not shown) that measures the individual's skin conductance. The method additionally analyzes the individual's skin conductance related to the auditory startle response to determine the likelihood that the individual is suffering from PTSD.

Individuals suffering from PTSD have demonstrated an elevated heart rate response to startling stimuli. These individuals have also shown larger skin conductance and orbicularis oculi EMG responses and slower skin conductance and EMG response habituation. Slower skin conductance habituation, determined by the number of stimulus trials required to reach a nonresponsive criterion, correlates with heightened autonomic conditionability. Differences in physiological response to startling tones develop along with PTSD in the months that follow a traumatic event.

The foregoing description of an illustrated embodiment of the invention has been presented for purposes of illustration and description, and is not intended to be exhaustive or to limit the invention to the precise form disclosed. The description was selected to best explain the principles of the invention and practical application of these principles to enable others skilled in the art to best utilize the invention in various embodiments and various modifications as are suited to the particular use contemplated. It is intended that the scope of the invention not be limited by the specification, but be defined by the claims set forth below.

What is claimed is:

1. A method for the biological diagnosis of Post-Traumatic Stress Disorder (PTSD) accomplished via a software application on an electronic device computer, comprising the steps of:
a. recording at least one of a first diameter of a pupil of an individual and a first video of a pupillary response of the individual using a camera in communication with the electronic device computer;
b. shining a light at the pupil of the individual using a light source in communication with the electronic device computer;
c. recording at least one of a second diameter of the pupil of the individual and a second video of a pupillary response of the individual after the application of the light source using the camera in communication with the electronic device computer;
d. determining at least one of a first difference in diameter between the first diameter and the second diameter and a second difference between the first video and the second video;
e. analyzing at least one of the first difference and the second difference based on a set of criteria of PTSD; and
f. determining a likelihood that the individual suffers from PTSD.

2. The method of claim 1, further comprising the steps of:
a. showing the individual an emotionally provocative visual stimuli;
b. recording at least one of a third diameter of the pupil of the individual and a third video of a pupillary response of the individual after showing the emotionally provocative visual stimuli using the camera in communication with the electronic device computer;
c. determining at least one of a third difference in diameter between the first diameter, second diameter, and third diameter and a fourth difference between the first video, second video, and third video;
d. analyzing at least one of the third difference and fourth difference based on a set of criteria of PTSD; and
e. determining a likelihood that the individual suffers from PTSD.

3. The method of claim 1, further comprising the steps of:
a. monitoring the heart rate of the individual continuously and simultaneously using at least one of the camera in communication with the electronic device computer and a heart rate monitor in communication with the electronic device computer;
b. determining a variability of the heart rate of the individual;
c. analyzing the variability; and
d. determining the likelihood that the individual suffers from PTSD.

4. A method for the biological diagnosis of Post-Traumatic Stress Disorder (PTSD) accomplished via a software application on an electronic device computer, comprising the steps of:
a. playing a first sudden, loud auditory sound to the individual using a head set in communication with the electronic device computer;
b. measuring a first orbicularis oculi electromyographic response of the individual using at least one electromyography lead in communication with the head set;
c. measuring a first heart rate of the individual using a heart rate monitor in communication with the head set;
d. playing a second sudden, loud auditory sound to the individual using the head set in communication with the electronic device computer;
e. measuring a second orbicularis oculi electromyographic response of the individual using at least one electromyography lead in communication with the head set;
f. measuring a second heart rate of the individual using a heart rate monitor in communication with the head set;
g. determining a variability of the heart rate of the individual based upon the first heart rate and the second heart rate;
h. determining an orbicularis oculi electromyographic response difference between the first orbicularis oculi electromyographic response and the second orbicularis oculi electromyographic response;
i. analyzing at least one of the variability and the orbicularis oculi electromyographic response difference based on a set of criteria of PTSD; and
j. determining the likelihood that the individual suffers from PTSD.

5. The method of claim 4, further comprising the steps of:
a. recording at least one of a first diameter of a pupil of the individual and a first video of a pupillary response of the individual using a camera in communication with the electronic device computer;
b. showing the individual an emotionally provocative visual stimuli;
c. recording at least one of a second diameter of the pupil of the individual and a second video of a pupillary response of the individual after showing the emotionally provocative visual stimuli using the camera in communication with the electronic device computer;
d. determining at least one of a first difference in diameter between the first diameter and the second diameter and a second difference between the first video and the second video;
e. analyzing at least one of the first difference and second difference based on a set of criteria of PTSD; and
f. determining a likelihood that the individual suffers from PTSD.

6. The method of claim 5, further comprising the steps of:
a. monitoring a third heart rate of the individual continuously and simultaneously using at least one of the camera in communication with the electronic device computer and a heart rate monitor in communication with the electronic device computer;
b. determining a second variability of the individual based upon at least one of the first heart rate, the second heart rate, and the third heart rate;
c. analyzing the second variability; and
d. determining the likelihood that the individual suffers from PTSD.

7. The method of claim 4, wherein the sudden, loud auditory sound is at least 95 decibels.

8. The method of claim 1, wherein the electronic device computer is a mobile electronic device.

9. The method of claim 4, wherein the electronic device computer is a mobile electronic device.

10. A system for the biological diagnosis of Post-Traumatic Stress Disorder (PTSD), comprising:
a. an electronic device computer;
b. a camera in communication with the electronic device computer, said camera communicating with the software application and adapted to capture at least one of still images and videos of an individual's pupillary response; and
c. a software application installed on the electronic device computer, said software application adapted to analyze camera data received from the camera.

11. The system of claim 10, further comprising a flash in communication with the electronic device computer, said flash communicating with the software application.

12. The system of claim 10, further comprising a heart rate monitor in communication with the electronic device computer, said heart rate monitor communicating with the software application and said software application adapted to analyze heart rate data received from the heart rate monitor.

13. The system of claim 10, further comprising a head set in communication with the electronic device computer, said head set communicating with the software application.

14. The system of claim 10, wherein the electronic device computer is a mobile electronic device.

15. The system of claim 10, wherein the camera is one of a built-in camera and an attachable camera.

16. The system of claim 11, wherein the flash is one of a built-in flash and an attachable flash.

17. The system of claim 12, wherein the heart rate monitor is one of a pulse oximeter probe, a digital finger monitor, and an ear lobe monitor.

18. The system of claim 13, wherein the head set includes a heart rate monitor.

19. The system of claim 13, wherein the head set includes at least one of at least one electromyography lead, at least one bispectral index lead, and at least one electroencephalogram lead.

20. The system of claim 18, wherein the heart rate monitor is a pulse oximeter probe.

21. The method of claim 4, further comprising the step of:
a. measuring at least one of the individual's depth of consciousness and the individual's depth of anesthesia using at least one of at least one bispectral index lead and at least one electroencephalogram lead in communication with the head set.

22. The system of claim 13, wherein the head set includes at least one of at least one disposable pulse oximeter probe, at least one disposable electromyography lead, and at least one disposable electroencephalogram lead.

23. The system of claim 13, wherein the head set includes at least one of at least one attachable pulse oximeter probe, at least one attachable electromyography lead, and at least one attachable electroencephalogram lead.

24. The system of claim 13, further comprising a kit that includes at least one of a disposable pulse oximeter probe, at least one electromyography lead, and at least one electroencephalogram lead.

25. The system of claim 13, wherein the head set includes a conductance sensor.

26. The method of claim 4, further comprising the steps of:
a. measuring a first skin conductance of the individual after the first sudden, loud auditory sound is played, said first skin conductance measured using a conductance sensor in communication with the head set;
b. measuring a second skin conductance of the individual after the second sudden, loud auditory sound is played, said second skin conductance measured using the conductance sensor in communication with the head set;
c. determining a skin conductance difference between the first skin conductance and the second skin conductance;
d. analyzing the skin conductance difference based on a set of criteria of PTSD; and
e. determining the likelihood that the individual suffers from PTSD.

27. The method of claim 4, further comprising the step of measuring a bispectral index of the individual using a bispectral index monitor in communication with the head set.

28. The method of claim 4, further comprising the step of measuring an electrical activity in the brain of the individual using at least one electroencephalogram lead in communication with the head set.

29. The method of claim 4, further comprising the steps of:
a. measuring a first electrical activity in the brain of the individual after the first sudden, loud auditory sound using at least one electroencephalogram lead in communication with the head set;
b. measuring a second electrical activity in the brain of the individual after the second sudden, loud auditory sound using at least one electroencephalogram lead in communication with the head set;
c. determining an electrical activity difference of the individual based upon the first electrical activity and the second electrical activity;
d. analyzing the electrical activity difference; and
e. determining the likelihood that the individual suffers from PTSD.
